(19) United States
(12) Patent Application Publication
(10) Pub. No.: US 2005/0187295 A1
(43) Pub. Date: Aug. 25, 2005

(54) Processes for the preparation of gabapentin

(76) Inventors: Surendra Kalyan, Andhra Pradesh (IN); Mysore Aswathanarayana Rao, Andhre Pradesh (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/852,743

(22) Filed: May 24, 2004

(30) Foreign Application Priority Data
Feb. 19, 2004 (IN) 127/CHE/2004

(51) Int. Cl. C07C 229/28; A61K 31/195

(52) U.S. Cl. 514/561; 562/507

(57) ABSTRACT

The present invention relates to a process of preparing gabapentin from its hydrochloride salt by the use of alkylamine and choice of solvent. The resulting gabapentin can be isolated either as Form II or Form III, characterized by their spectra, by simply choosing an appropriate solvent in the process.

### FIELD OF INVENTION

The present invention relates to a process for the preparation of gabapentin. The invention more particularly relates to a process for the conversion of gabapentin hydrochloride salt to gabapentin.

### BACKGROUND OF THE INVENTION

Gabapentin is chemically, 1-(aminomethyl)-1-cyclohexane acetic acid, having the chemical formula I structure shown below (The Merck Index, 13^{th} Ed., page 767, No.4342):

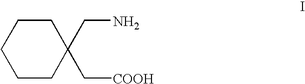

Gabapentin is therapeutically useful in treating epilepsy and various other cerebral disorders. It was first described by Warner-Lambert Co. in U.S. Pat. No. 4,024,175.

Several processes for the preparation of gabapentin are reported in the literature. U.S. Pat. No. 4,024,175 only indicates in one of the examples that “by treatment with a basic ion exchanger and crystallization from ethanol/ether, there is obtained pure 1-aminomethyl-1-cyclohexane acetic acid . . . ”, without any further details or claims. U.S. Pat. No. 5,091,567 discloses the conversion of the gabapentin hydrochloride salt to gabapentin base by passing it over a column filled with a weakly basic anion exchanger, without giving any further details about the preferred ion exchanger or how to achieve this (see example 10 in this patent). U.S. Pat. No. 4,894,476 discloses a hydrated form of gabapentin which is prepared by liberating the free gabapentin base from its hydrochloride salt by pouring the salt solution onto a column of ion exchange resin like Amberlite IRA-68, eluting with deionised water and further work up to recover the hydrated form. WO 02/34709 describes the use of strong cation exchange resins like IMAC HP1110, IRA 120 etc., for converting the gabapentin hydrochloride to gabapentin base. The process requires a double elution, first to remove anions and then the cations. These processes generally result in obtaining “Form II” which is commercially marketed.

Column chromatography is not convenient for applications on an industrial scale. It requires long time and results in a large volume of aqueous solution to be evaporated at low temperature, which makes the process uneconomical.

Diafiltration technique for the separation of salt from gabapentin is described in the WO 00/58268 (equivalent to the U.S. Pat. No. 6,576,790). The technique uses a nanofiltration multilayer composite membrane having selectivity for organic compounds with molecular weight higher than 150 and low selectivity to inorganic ions.

The drawback of this process is that these membranes are not easily available and are also expensive. Further, special reactors are required for carrying out the process.

A process for the conversion of gabapentin hydrochloride to free gabapentin is described in the WO 98/28255. In this process gabapentin hydrochloride is dissolved in a solvent such as ethyl acetate, in which free gabapentin is insoluble. An alkylamine such as tributylamine, is added to the solution resulting in the precipitation of free gabapentin, which is insoluble. Alkylamine hydrochloride formed in the reaction, being soluble, will remain in solution. Free gabpentin is recovered by filtration.

There are several drawbacks in the process as described in WO 98/28255. Since gabapentin precipitates directly from the solvent, it is obtained as a polymorph, which is different from the commercially marketed polymorph (Form II). An additional step involving treatment with methanol is required to convert the new polymorph (called Form III by the inventors) into the commercially marketed Form II. The alkylamine used is selected from the group consisting of triethylamine, tributylamine, tripropylamine, trihexylamine, diethylamine, ethanolamine, and benzylamine as described in the patent WO 98/28255. All these amines are nonvolatile liquids at ambient temperature and difficult to remove when present in excess, requiring repeated extractions. Gabapentin also has a free carboxylic acid group, which can form salt with excess amine. Such salts are not desirable in the final product.

### SUMMARY OF THE INVENTION

The present invention is a novel process and provides an industrially useful process for the conversion of gabapentin hydrochloride to gabapentin in a commercially marketable form of polymorph (Form II) directly in a one pot reaction. The process consists of using a solvent such as methanol in which both gabapentin hydrochloride and gabapentin are soluble and passing through the solution a gaseous alkylamine such as dimethylamine, which liberates free gabapentin from its hydrochloride salt. The excess of the alkylamine is easily removed by heat or flushing during the process. On removing the solvent by distillation, a residue is obtained which is washed with a solvent that removes the alkylamine hydrochloride formed during the reaction, leaving behind the required polymorphic form of gabapentin. By using isopropanol or chloroform as a wash solvent, the Form II polymorph is obtained. By using isoamyl alcohol instead as wash solvent, the Form III polymorph is obtained. Thus, by the same process, it is possible to obtain either Form II (currently commercially marketed) or Form III (as described in WO 98/28255) simply by changing the wash solvent, directly from the gabapentin hydrochloride without isolation and further processing.

### DETAILED DESCRIPTION OF THE INVENTION

The present invention provides for process for the conversion of gabapentin hydrochloride to gabapentin polymorphic Form II, which comprises
(i) dissolving gaseous alkylamine or a solution of the alkylamine in methanol in which free gabapentin is also soluble;
(ii) passing gaseous alkylamine or a solution of the alkylamine in methanol to the resulting solution of step (i), till the pH is neutral at ambient temperature; and
(iii) removing the solvent employing low pressure and washing the residue with a solvent in which the alkylamine hydrochloride formed during the reaction is soluble and isolating the free gabapentin of desired polymorphic form.

Gabapentin hydrochloride can be prepared by one of the methods described in the literature, for example, U.S. Pat. Nos. 4,024,175 or 4,152,326, incorporated by reference herein. It is dissolved in methanol in which free gabapentin is also soluble. The selection of the solvent is important, because if the free gabapentin is not soluble, the polymorphic form obtained will be different from that of the commercially marketed form and an additional step will be required to convert the product into a commercially marketable form.

The second step of the present invention comprises passing an alkylamine, which is in the gaseous form at ambient temperature to the solution of gabapentin hydrochloride. Alkylamines, which are gaseous at ambient temperature, are for example, methylamine, dimethylamine and trimethylamine and other alkylamines. Preferred alkylamines are dimethylamine and trimethylamine because their hydrochloride salts are soluble in a number of organic solvents such as methanol, isopropanol, isoamyl alcohol, chloroform, etc. The alkylamine may also be added as a solution in methanol. These alkylamines being highly reactive, convert gabapentin hydrochloride to gabapentin rapidly. When the pH reaches 7.0 to 7.5, from the initial pH of about 1.5, addition of the amine is discontinued. At this stage the solution remains clear. The solvent is removed at a reduced pressure. Excess alkylamine, if any, will also be eliminated at this stage. The residue obtained is again dissolved in methanol and concentrated to small volume to precipitate gabapentin. It is cooled and filtered. The precipitate is washed repeatedly with small quantities of solvent in which the amine hydrochloride is soluble. The solvents preferred are isopropanol or chloroform. Methanol can also be used in small quantities and at cold temperatures, keeping in mind that gabapentin is soluble in methanol. Methanol removes amine hydrochlorides efficiently. Washing is repeated till the filtrate is negative to chloride as indicated by silver nitrate test. At this stage the residue is also negative to silver nitrate test. The residue is dried under vacuum. It exhibits the peaks in the X-ray diffraction diagram with 2-theta values at 6.0, 7.8, 14.9, 16.8, 20.2, 23.5, 26.7, 28.1 degrees which are characteristic of the Form II.

Interestingly, when isoamyl alcohol is used as a solvent to remove dimethylamine hydrochloride, the material obtained is found to be of different polymorph, named as Form Ell. The X-ray diffraction pattern and infra-red spectra of this polymorph are same as that of the novel form disclosed in WO 98/28255.

### ADVANTAGES OF THE INVENTION

1). The process is simple and can be applied commercially

2). The process does not use ion exchange resins and tedious column chromatography.

3). The alkyl amine used can be easily removed

4). The process can yield either commercially marketed Form II or the Form III by a simple change of solvent.

The embodiments of the present invention are further described in the following examples, which are not intended in any way to limit the scope of the present invention.

### EXAMPLE 1

10.0 g of gabapentin hydrochloride were dissolved in 100 mls dry methanol. Dimethylamine gas was bubbled into the solution till the pH was 7 to 7.5. After stirring for 30 minutes, most of the solvent was removed under reduced pressure, the thick slurry was filtered to obtain 11.5 gms precipitate. The precipitate was suspended in 15 mls dry methanol, stirred for 10 minutes and filtered. The process was repeated until the filtrate was negative to chloride as shown by AgNO₃ test. The residue was dried under vacuum to obtain 3.6 g of gabapentin. From the pooled filtrate a second crop of 0.9 g pure material can be obtained. Total yield: 4.5 g (54.6%), m.p: 160-161° C., purity by HPLC: 99.1%. It displayed a characteristic X-ray diffraction pattern with 2-theta values at 6.0, 7.8, 14.9, 16.8, 20.2, 23.5, 26.7, and 28.1 degrees and characteristic infra-red absorption peaks at 708.6, 749.0, 890.6, 927.9, 976.1, 1165.1, 1300.1, 1420.2, 1474.9, 1543.4, and 1614.9 cm⁻¹. These X-ray diffraction patterns and infra-red peaks are characteristics of Form II.

### EXAMPLE 2

10.0 g of gabapentin hydrochloride were dissolved in 100 mls dry methanol. Dimethylamine gas was bubbled into the solution till pH was 7 to 7.5. After stirring for 30 minutes, most of the solvent was removed under reduced pressure, the thick slurry was filtered to obtain 11.5 gms precipitate. The precipitate was suspended in 15 mls dry isopropanol, stirred for 10 minutes and filtered. The process was repeated till the filtrate was negative to chloride as shown by AgNO₃ test. The residue was dried under vacuum to obtain 4.2 gms of gabapentin. From the pooled filtrate, a second crop of 0.8 gms pure material can be obtained. Total yield: 5.0 gms (60.6%), melting point: 160-162° C., purity by HPLC: 99.3%. It displayed characteristic X-ray diffraction pattern and infra-red peaks as given in the Example 1.

### EXAMPLE 3

10.0 g of gabapentin hydrochloride were dissolved in 100 mls dry methanol. Dimethylamine gas was bubbled into the solution till pH was 7 to97.5. After stirring for 30 minutes, most of the solvent was removed under reduced pressure, the thick slurry was filtered to obtain 11.5 grams precipitate. The precipitate was suspended in 15 mls dry isoamyl alcohol, stirred for 10 minutes and filtered. The process was repeated till the filtrate was negative to AgNO₃ test. The residue was dried under vacuum to obtain 4.0 gms (48.5%) of gabapentin. Melting point: 156-158° C., purity by HPLC: 99.2%. It displayed a characteristic X-ray diffraction pattern with 2-theta values at 6.02, 12.07, 24.35, 5.60, 16.84, 11.84, 17.99, and 20.64 degrees (decreasing order of the peak size with the peak at 6.02 degree=100%) and characteristic infra-red absorption peaks at 708.6, 749.0, 890.6, 927.9, 976.1, 1165.1, 1300.1, 1420.2, 1474.9, 1543.4, and 1614.9 cm⁻¹. These X-ray diffraction patterns and infra-red peaks are characteristics of Form III (the novel polymorph as described in WO 98/28255).

### EXAMPLE 4

10.0 g of gabapentin hydrochloride were dissolved in 100 mls dry methanol. Dimethylamine gas was bubbled into the solution till pH was 7 to 7.5. After stirring for 30 minutes, most of the solvent was removed under reduced pressure, the thick slurry was filtered to obtain 11.5 gms precipitate. The precipitate was suspended in 15 mls dry chloroform, stirred for 10 minutes and filtered. The process was repeated six times to make the final filtrate negative to AgNO₃ test. The residue was dried under vacuum to obtain 6.0 grams (72.8%) of gabapentin Form II melting point: 160-162° C. Purity by HPLC: 98.9%. It displayed a characteristic X-ray diffraction pattern and infra-red peaks as given in Example 1.

### EXAMPLE 5

10.0 g of gabapentin hydrochloride were dissolved in 100 mls dry methanol. Trimethylamine gas was bubbled into the solution till pH was 7 to 7.5. After stirring for 30 minutes, most of the solvent was removed under reduced pressure, the thick slurry was filtered to obtain 11.9 grams precipitate. The precipitate was suspended in 15 mls dry isopropanol, stirred for 10 minutes and filtered. The process was repeated until the filtrate was negative to AgNO₃ test. The residue was dried under vacuum to obtain 2.9 grams of gabapentin. From the pooled methanolic filtrate a second crop of 0.8 g pure gabapentin Form II is obtained. Total yield: 3.7 g (45.0%), melting point: 160-162° C. Purity by HPLC: 99.1%. It displayed a characteristic X-ray diffraction pattern and infra-red peaks as in the Example 1.

1. A process for the conversion of gabapentin hydrochloride to gabapentin polymorphic Form II which comprises the steps of:
(i) dissolving gabapentin hydrochloride in methanol in which free gabapentin is also soluble to form a solution;
(ii) passing a gaseous alkylamine or a solution in methanol to the solution of step (i) until the pH is neutral at ambient temperature; and
(iii) removing the solvent from the solution of step (ii), by employing a pressure of less than 10 mm and washing the residue with a solvent in which the alkylamine hydrochloride formed in the reaction is soluble, to yield gabapentin polymorphic Form II or gabapentin polymorphic Form III by choosing an appropriate solvent for washing.

2. The process of claim 1 wherein the alkyl amine used in step (ii) is selected from the group consisting of methylamine, dimethylamine, and trimethylamine.

3. The process of claim 1 wherein the solvent used to remove the alkylamine hydrochloride in step (iii) is selected from the group consisting of methanol, isopropanol, or chloroform, to yield gabapentin polymorphic Form II.

4. The process of claim 1, wherein the solvent used to remove the alkylamine hydrochloride in step (iii) is isoamyl alcohol, to yield gabapentin polymorphic Form III.

5. A process for the conversion of gabapentin hydrochloride to gabapentin polymorphic Form II substantially as herein described with reference to the Examples 1, 2, 4 & 5.

6. A process for the conversion of gabapentin hydrochloride to gabapentin polymorphic Form III substantially as herein described with reference to the Example 3.
